(19) United States
(12) Patent Application Publication
(10) Pub. No.: US 2009/0163697 A1
(43) Pub. Date: Jun. 25, 2009

(54) Multicomponent coupling and glycopeptide synthesis with cyclic thioanhydrides

(76) Inventors: David Crich, Grosse Point Park, MI (US); Albert A. Bowers, Somerville, MA (US)

Correspondence Address: Olson & Cepuritis, LTD., 20 NORTH WACKER DRIVE, 36TH FLOOR, CHICAGO, IL, 60606, US

(21) Appl. No.: 12/317,139

(22) Filed: Dec. 19, 2008

Related U.S. Application Data
(60) Provisional application No. 61/015,343, filed on Dec. 20, 2007.

(51) Int. Cl. C07K 1/107; C07H 1/00; C07D 279/16; C07C 227/18; C07D 295/15

(52) U.S. Cl. 530/345; 536/55.3; 544/165; 544/52; 562/561

(57) ABSTRACT

Disclosed is a method of coupling an amino or hydroxyl compound with the amino portion of a sulfonamide via condensation with a cyclic thioanhydride. The reaction of cyclic thioanhydrides with amines affords amides functionalized with thioacids, which can be trapped in situ with preferably electron deficient arylsulfonamides. In this manner the cyclic thioanhydride serves as a linchpin in a three component coupling sequence.

### CROSS-REFERENCE TO RELATED APPLICATION

This application claims the benefit of U.S. Provisional Application for Patent Ser. No. 61/015,343, filed on Dec. 20, 2007, which is incorporated herein by reference in its entirety.

### FIELD OF THE INVENTION

This invention relates to organic coupling reactions, and more particularly to methods of coupling amino and hydroxyl compounds.

### BACKGROUND

Thioacids are useful intermediates in organic synthesis, having found application in peptide synthesis, and as precursors to thioesters for native chemical ligation. Their long-established reaction with azides yields secondary amides and has come to the fore as a versatile ligation reaction, while their complementary reaction with 2,4-dinitrobenzenesulfonamides has been much less widely applied. The relatively limited application of these useful coupling reactions can be ascribed, at least in part, to the need to prepare all but the simplest thioacids. Formation of aryl sulfonamide reagents from amines is well known in the art.

It is often useful or necessary in the pharmaceutical arts to prepare compounds that include two different functional materials, e.g., a tissue specific targeting agent and a pharmaceutically useful group, such as a drug or other chemotherapeutic agent; or glycopeptides, which comprise a polypeptide material coupled to a sugar or polysaccharide. In many cases, the different functional materials include an amino group, or can be prepared in a form in which at least one of the materials comprises an amino group, and the other material includes or can be functionalized with either an amino group or a hydroxyl group.

The methods of the present invention fulfill the need for such coupling methods through the nucleophilic ring opening of cyclic thioanhydrides with the in situ generation of a thioacid for use in a coupling reaction. Limited precedent is provided by the opening of thiosuccinic anhydride with amines with subsequent trapping of the thioacid with benzyl bromide, providing a thioester for subsequent use in native chemical ligation. The present facile synthesis and stability of cyclic thioanhydrides provides for a new method of multicomponent coupling reactions.

### SUMMARY OF THE INVENTION

The reaction of cyclic thioanhydrides with amines affords amides functionalized with thioacids, which can be trapped in situ with electron deficient arylsulfonamides, preferably 2,4-dinitrobenzenesulfonamides. In this manner the cyclic thioanhydride serves as a linchpin in a three component coupling sequence between an amine or alcohol and a sulfonylated amine. The use of thiomaleic anhydride and a bifunctional nucleophile including both an imine and a thiol extends the process to heterocycle synthesis, while cyclic thioanhydrides derived from aspartic acid and glutamic acid directly provide N-functionalized asparagine and glutamine derivatives, respectively.

In particular, the reaction of readily available cyclic thioanhydrides with an amine or alcohol and an electron deficient arylsulfonamide (e.g., a 2,4-dinitrobenzenesulfonamide) represents a very efficient multicomponent coupling sequence employing the cyclic thioanhydride as the linchpin and ultimate source of a linking group between an amine or alcohol and the amino portion of a sulfonamide. The use of unsaturated cyclic thioanhydrides and bifunctional nucleophiles (e.g., an aminothiol) provides a multicomponent heterocycle synthesis, while amino acid-based cyclic thioanhydrides have the potential to serve as linchpins in the convergent synthesis of glycopeptides.

In one embodiment, the present invention provides a method for coupling an amino or hydroxyl compound with the amino portion of a sulfonamide. The method comprises contacting an amino or hydroxyl compound of Formula (I) with a cyclic thioanhydride of Formula (II) for a period of time sufficient to form a thioacid intermediate of Formula (III), and contacting the intermediate of Formula (III) with an aryl sulfonamide compound of Formula (IV) in the presence of a base to form a coupled amide of Formula (V), as set forth in the reaction scheme shown in FIG. 1. In the structural formulas shown in FIG. 1, X¹ is —NH— or —O—; Y¹ is a hydrocarbon group forming a 4- to 10-membered ring with the thioanhydride portion of Formula II; Ar¹ is an electron deficient aryl group; and R¹ and R² are each independently a hydrocarbon group, a carbohydrate group, an amino acid group, or a peptide group.

Non-limiting examples of cyclic thioanhydrides useful in the methods of the present invention are shown in FIG. 2. In the structural formulas shown in FIG. 2, R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; each R^{c} is independently H, alkyl, arylalkyl, or aryl; x is 1, 2, 3, 4, 5, or 6; each R^{d} is independently H, alkyl, arylalkyl, aryl, or both R^{d} groups together form —CR^{c}₂—(CR^{c}₂)_{z}—CR^{c}₂—; z=1, 2, 3 or 4, each X^{a} independently is H, alkyl, arylalkyl, aryl, halogen, nitro, CN, fluoroalkyl, acyl, amino, hydroxyl, alkoxy, arylalkoxy, aryloxy, alkylamino, arylalkylamino, or arylamino; and p=1, 2, 3 or 4.

In another embodiment, the compound of Formula (I) comprises an aminothiol compound. In this embodiment, the present invention provides a method for coupling the aminothio compound with a sulfonated amino compound to form a sulfur-nitrogen heterocycle. The method comprises contacting an aminothio compound of Formula (29) with a cyclic thioanhydride of Formula (24) for a period of time sufficient to form a thioacid intermediate, e.g., a compound of Formula (30), and contacting the thioacid intermediate with an aryl sulfonamide compound of Formula (IV) in the presence of a base to form a coupled heterocyclic amide of Formula (31), as set forth in the reaction scheme shown in FIG. 3. In the structural formulas shown in FIG. 3, R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; Ar¹ is an electron deficient aryl group; R² is a hydrocarbon group, a carbohydrate group, an amino acid group, or a peptide group; and wherein each R^{f}, R^{g}, R^{h}, and Rⁱ independently is H, alkyl, arylalkyl, aryl, or alternatively, R^{f} and R^{g} are H, while R^{h} and Rⁱ together form an aliphatic hydrocarbon ring; or R^{f} and R^{g} are absent, while R^{h} and Rⁱ together form an aromatic hydrocarbon ring.

In yet another embodiment, the cyclic thioanhydride compound is a protected aspartic acid-derived or protected glutamic acid-derived thioanhydride. This embodiment provides a method for coupling an amino or hydroxyl compound with the thioanhydride and an aryl sulfonamide to form an asparagine or glutamine derivative, as the case may be. This method comprises contacting an amino or hydroxyl compound of Formula (I) with a cyclic thioanhydride of Formula (26) for a period of time sufficient to form an intermediate of Formula (32), and contacting the intermediate of Formula (32) with an aryl sulfonamide compound of Formula (IV) in the presence of a base to form a coupled asparagine or glutamine derivative of Formula (33), as set forth in the reaction scheme shown in FIG. 4. In the structural formulas shown in FIG. 4, X¹ is —NH— or —O—; Ar¹ is an electron deficient aryl group; R¹ and R² are each independently a hydrocarbon group, a carbohydrate group, an amino acid group, or a peptide group; R^{b} is H, alkyl, arylalkyl, aryl, alkyloxy, arylalkyloxy, aryloxy, alkylamino, arylalkylamino, arylamino, or NH₂; and y is 1 or 2.

The methods of the present invention are useful for a variety of purposes. In particular, the present methods provide a facile means for coupling an amino and hydroxyl compound with a sulfonated amine, with subsequent loss of the sulfonyl group, to afford bisamide or ester-amide products, depending respectively on whether an amine or alcohol is condensed with the thioanhydride. Accordingly, the methods of the present invention beneficially can be used, for example, to readily couple an amino or hydroxyl substituted pharmaceutically useful group (e.g., a tissue targeting agent) to an arylsulfonamide substituted pharmaceutically useful group (e.g., a chemotherapeutic agent). The methods can also be used for the preparation of glycopeptides. In this context, the use of cyclic aspartic or glutamic thioanhydrides is particularly useful, since the resulting product can link a peptide to a polysaccharide (e.g., a glycoside) via an asparagine or glutamine linking group. Beneficially, the methods of the present invention are performed under conditions that are compatible with a wide variety of chemical structures and functional groups.

### BRIEF DESCRIPTION OF THE DRAWINGS

FIG. 1 schematically illustrates the method of this invention for coupling amino and hydroxyl compounds via condensation with a cyclic thioanhydride; X¹ is —NH— or —O—; Y¹ is a hydrocarbon group forming a 4- to 10-membered ring with the thioanhydride portion of Formula II; Ar¹ is an electron deficient aryl group; and R¹ and R² are each independently a hydrocarbon group, a carbohydrate group, an amino acid group, or a peptide group.

FIG. 2 illustrates certain cyclic thioanhydride compounds useful in the methods of the present invention; R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; each R^{c} is independently H, alkyl, arylalkyl, or aryl; x is 1, 2, 3, 4, 5, or 6; each R^{d} is independently H, alkyl, arylalkyl, aryl, or both R^{d} groups together form —CR^{c}₂—(CR^{c}₂)_{z}—CR^{c}₂—; z=1, 2, 3 or 4, each X^{a} independently is H, alkyl, arylalkyl, aryl, halogen, nitro, CN, fluoroalkyl, acyl, amino, hydroxyl, alkoxy, arylalkoxy, aryloxy, alkylamino, arylalkylamino, or arylamino; and p=1, 2, 3 or 4.

FIG. 3 schematically illustrates an embodiment of the method of the present invention useful for forming a nitrogen-sulfur heterocyclic compound; R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; Ar¹ is an electron deficient aryl group; R² is a hydrocarbon group, a carbohydrate group, an amino acid group, or a peptide group; and wherein each R^{f}, R^{g}, R^{h}, and Rⁱ independently is H, alkyl, arylalkyl, aryl, or alternatively, R^{f} and R^{g} are H, while R^{h} and Rⁱ together form an aliphatic hydrocarbon ring; or R^{f} and R^{g} are absent, while R^{h} and Rⁱ together form an aromatic hydrocarbon ring.

FIG. 4 schematically illustrates an embodiment of the method of the present invention useful for forming asparagine and glutamine derivatives; X¹ is —NH— or —O—; Ar¹ is an electron deficient aryl group; R¹ and R² are each independently a hydrocarbon group, a carbohydrate group, an amino acid group, or a peptide group; R^{b} is H, alkyl, arylalkyl, aryl, alkyloxy, arylalkyloxy, aryloxy, alkylamino, arylalkylamino, arylamino, or NH₂; and y is 1 or 2.

FIG. 5 illustrates, in tabular form, a series of eight examples of three-component coupling reactions of this invention conducted via condensation of cyclic thioanhydrides with amines and 2,4-dinitrobenzenesulfonamides.

### DETAILED DESCRIPTION OF PREFERRED EMBODIMENTS

As used herein, the term “hydrocarbon” broadly encompasses any organic molecule containing one or more carbon atoms. Hydrocarbon moieties be can substituted and unsubstituted aliphatic groups, as well as substituted and unsubstituted aryl groups, which groups can be saturated or can include one or more sites of unsaturation (e.g., a double bond, triple bond, or aromatic group). Hydrocarbons can comprise, consist of, or consist essentially of branched chains, linear chains, or cyclic chains of carbon atoms, as well as any combination thereof. The term “aryl” refers to aromatic hydrocarbon groups (e.g., phenyl, naphthyl, and the like), as well as heteroaromatic hydrocarbon groups (i.e., aromatic moieties including a heteroatom such as nitrogen, oxygen, or sulfur within an aromatic ring, such as a pyridine group, a furan group, a thiophene group, and the like). The term “unsubstituted” refers to a hydrocarbon moiety in which each carbon atom is bound only to another carbon or a hydrogen. The term “substituted” refers to a hydrocarbon moiety in which at least one carbon atom is bound to a heteroatom (i.e., an atom other than carbon or hydrogen, e.g., nitrogen, oxygen, sulfur, phosphorus, a halogen, and the like). Substituted hydrocarbon moieties encompass materials having a heteroatom bound to a single carbon atom or to multiple carbon atoms. Such heteroatoms can be appended along a chain of carbon atoms (e.g., as in an alcohol), or within a chain (e.g., as in an ether).

The term “carbohydrate” refers to individual sugars, as well as dimers, oligomers, and polymers comprising multiple sugars linked together in linear or branched chains. Carbohydrates include sugars and polysaccharides, per se, and derivatives thereof, such as reduced, oxidized, esterified, and alkylated forms thereof. The term “amino acid” encompasses natural amino acids found in proteins, and non-protein-derived amino carboxylic acids, as well as derivatives thereof (e.g., esterified, and alkylated derivatives). The term “peptide” refers to a chain of two or more amino acids bound together by an amide bond between an amine group of one amino acid and a carboxylic acid group of another amino acid.

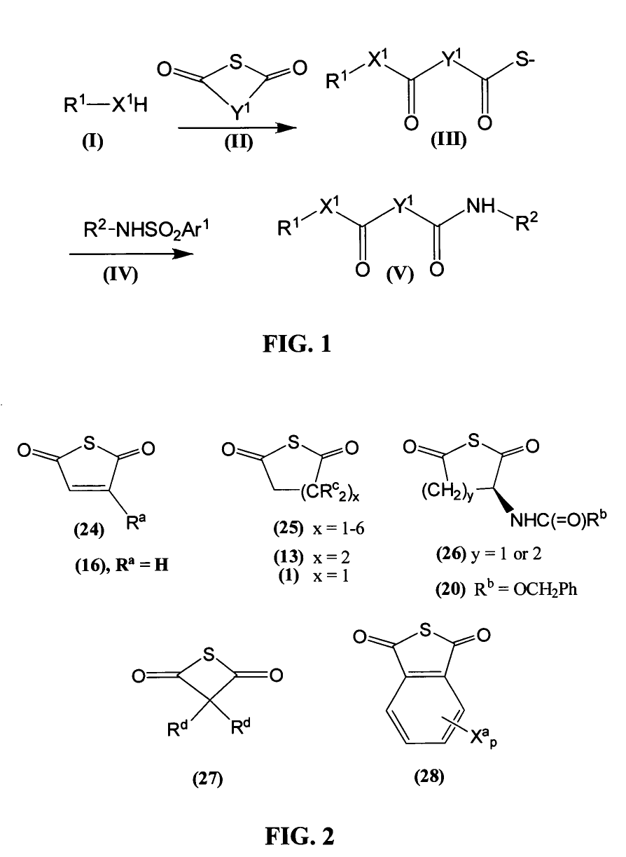

The present invention provides a method for coupling an amino or hydroxyl compound with the amino portion of a sulfonamide, which is schematically outlined FIG. 1. In the present methods, amino or hydroxyl compound (I) is contacted and condensed with a cyclic thioanhydride (II) for a period of time sufficient to form a thioacid intermediate thioacid (III) in which one carbonyl of the thioanhydride has been condensed with the amine or alcohol to respectively form an amide or ester therefrom. The intermediate thioacid (III) is contacted with a an electron deficient arylsulfonamide compound (i.e., IV) The thioacid (III) couples with sulfonamide (IV) in the presence of a base to form a product (V) in which the carbonyl group of the thioacid is coupled with the amine component of the sulfonamide, with loss of the arylsulfonyl group.

In thioanhydrides of Formula (II), certain preferred Y¹ groups include simple aliphatic or aromatic hydrocarbon groups that form a 4 to 10 member ring with the (O═C)—S—(C═O) portion of the thioanhydride, preferably a 4 to 6 membered ring. The hydrocarbon groups can be branched or unbranched, and can optionally be substituted with various functional groups. Other preferred Y¹ groups are derived from N-protected aspartic acid or N-protected glutamic acid derivatives. In some embodiments, the cyclic thioanhydride (II) is one of the thioanhydride compounds illustrated in FIG. 2, described above. Compounds of Formula (24) and (26) in FIG. 2 are particularly useful in certain embodiments of the present invention, which are described in more detail elsewhere herein.

The group R¹ in compounds of Formulas (I) and (III) can comprises any useful organic moiety. In certain preferred embodiments, R¹ comprises an amino acid group. In other preferred embodiments, R¹ comprises a peptide group (e.g., a polypeptide). In yet other embodiments, R¹ comprises a carbohydrate group (e.g., a sugar or a polysaccharide).

Similarly, the group R² in compound of Formulas (IV) and (V) also can comprises any useful organic moiety. In certain preferred embodiments, R² comprises an amino acid group. In other preferred embodiments, R² comprises a peptide group (e.g., a polypeptide). In yet other embodiments, R² comprises a carbohydrate group (e.g., a sugar or a polysaccharide). Preferably, X¹ in Formulas (I), (III), and V) is —NH—.

In some preferred embodiments of the compounds of Formula (IV), Ar¹ comprises an electron deficient substituted phenyl group. A particularly preferred Ar¹ is 2,4-dinitrophenyl.

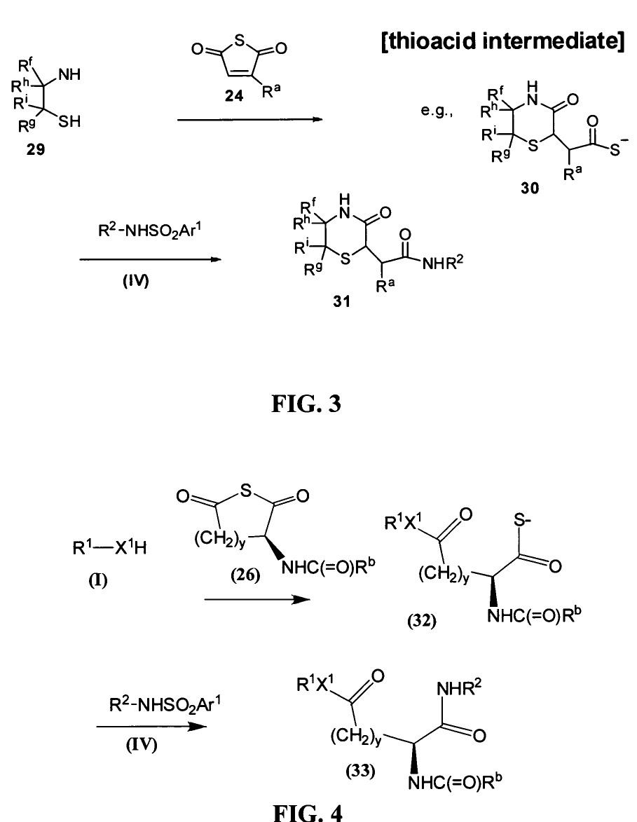

Another embodiment of the present invention provides a method for coupling an aminothio compound and the amino portion of a sulfonamide to form a sulfur-nitrogen heterocycle. In this embodiment, schematically illustrated in FIG. 3, an aminothio compound of Formula (29) is contacted with a unsaturated cyclic thioanhydride of Formula (24) for a period of time sufficient to form an intermediate thioacid, such as a compound of Formula (30). The intermediate thioacid is then contacted with an aryl sulfonamide compound of Formula (IV) in the presence of a base to form a coupled heterocyclic amide of Formula (31). In the reaction scheme illustrated in FIG. 3, the thiol group of aminothiol (29) nucleophilically adds to the olefin portion of unsaturated thioanhydride (24), and the amino group of aminothiol (29) condenses with a carbonyl of the thioanhydride thioacid (30), thus forming the heterocyclic amide. The Michael addition reaction of the thiol to the olefin may occur before, during, or after the reaction with the sulfonamide.

In the compounds of FIG. 3, R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; Ar¹ is an electron deficient aryl group; R² is a hydrocarbon group, a carbohydrate group, an amino acid group, or a peptide group; and wherein each R^{f}, R^{g}, R^{h}, and Rⁱ independently is H, alkyl, arylalkyl, aryl, or alternatively, R^{f} and R^{g} are H, while R^{h} and Rⁱ together form an aliphatic hydrocarbon ring; or R^{f} and R^{g} are absent, while R^{h} and Rⁱ together form an aromatic hydrocarbon ring.

Preferably, Ar¹ is 2,4-dinitrophenyl. In some preferred embodiments, R^{f} and R^{g} are absent, while R^{h} and Rⁱ together form an aromatic hydrocarbon ring. One example of such an embodiment utilizes 2-thioaniline as the aminothiol. This method beneficially provides a synthesis of 6-membered-ring nitrogen-sulfur heterocycles (i.e., having a sulfur in the 1 position and a nitrogen in the 4 position of the heterocyclic ring).

In yet another preferred embodiment, the present invention provides a method for coupling an amino or hydroxyl compound with an aspartic acid-derived or glutamic acid-derived thioanhydride and an aryl sulfonamide to form an asparagine or glutamine derivative, as the case may be. This method, illustrated in FIG. 4, comprises contacting an amino or hydroxyl compound of Formula (I) with a cyclic thioanhydride of Formula (26) for a period of time sufficient to form an intermediate of Formula (32), and contacting the intermediate of Formula (32) with an aryl sulfonamide compound of Formula (IV) in the presence of a base to form a coupled asparagine or glutamine derivative of Formula (33).

In the structural formulas shown in FIG. 4, X¹ is —NH— or —O—; Ar¹ is an electron deficient aryl group; R¹ and R² are each independently a hydrocarbon group, a carbohydrate group, an amino acid group, or a peptide group; R^{b} is H, alkyl, arylalkyl, aryl, alkyloxy, arylalkyloxy, aryloxy, alkylamino, arylalkylamino, arylamino, or NH₂; and y is 1 or 2. This method is particularly useful for preparing glycopeptides, in which case, at least one of R¹ and R² comprises a peptide moiety, and the other of R¹ and R² comprises a carbohydrate moiety (e.g., a sugar or polysaccharide). As in the other embodiments, it is preferred that the electron deficient aryl group, Ar¹ is an electron deficient phenyl group, e.g., a 2,4-dinitrophenyl group.

We have observed that the reaction of thiosuccinic anhydride (1) with benzylamine in methanol at room temperature, followed by addition of tosyl azide and 2,6-lutidine, affords a dissymmetric succinamide (2) in a quantitative yield of 100% (Scheme 1). In addition, thiosuccinic anhydride (1) was treated with free glycine as the initiating nucleophile in methanol, followed by the addition of tosyl azide and sodium bicarbonate (2 equivalents), to provide an excellent yield (86%) of dissymmetric succinamide (4) (Scheme 1). The preparation of thioanhydrides is described by Kates and Schauble in J. Org. Chem., 60, 6676-6677 (1995) and in J. Heterocycl. Chem., 32, 971-978 (1995), the relevant disclosures of which are incorporated herein by reference.

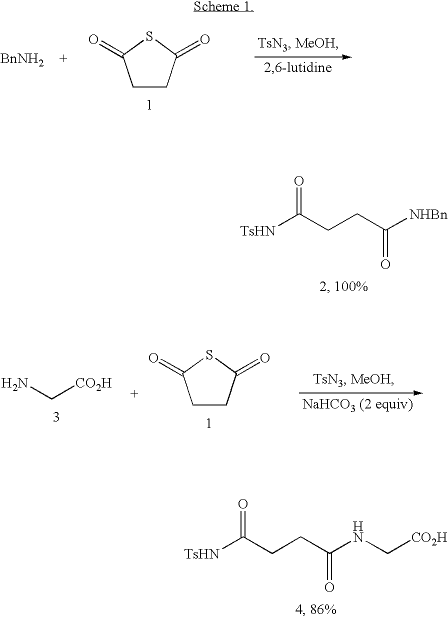

The reaction sequences of the present invention utilize an activated, electron deficient aryl sulfonamide (e.g., a 2,4-dinitrobenzenesulfonamide) in place of tosyl azide, to provide a three component coupling method. The present methods provide a versatile coupling procedure that is applicable to a variety of amines and sulfonamides, and which has the capability for synthesis of tertiary amides. Beneficially, the coupling reaction of the present invention avoids the use of toxic azide compounds.

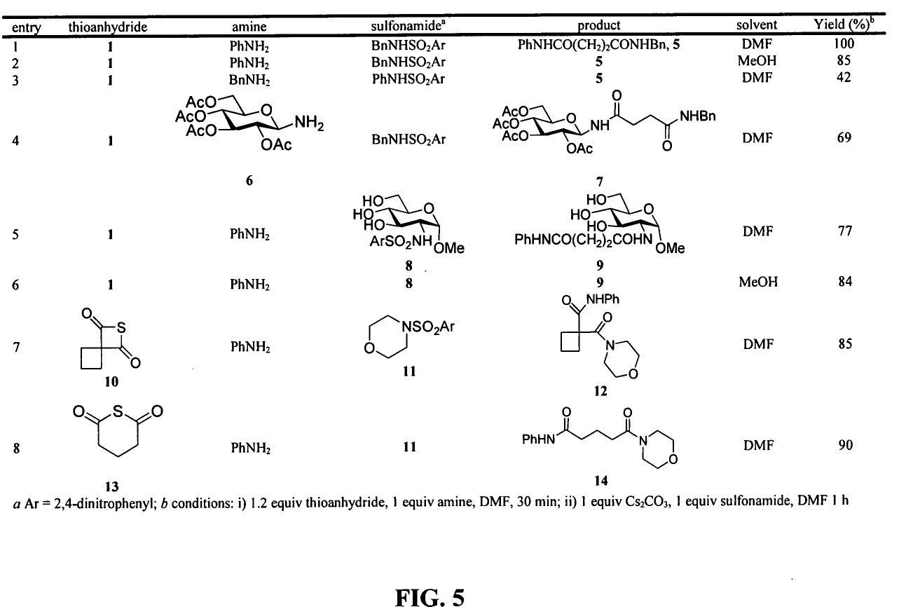

FIG. 5 presents, in tabular form, a series of eight examples conducted in the three component method of the invention, each of which afforded excellent yields of the products. All reactions in FIG. 5 were conducted under ambient conditions by addition of the nucleophilic amine to the cyclic thioanhydride, followed by addition of the sulfonamide and a mild base, Cesium carbonate (CS₂CO₃); and the overall reaction time, including work-up was less than two hours. The reaction sequence is not restricted to the opening of five-membered cyclic thioanhydrides, and is also suitable for larger ring sizes, such as thiomalonic anhydride and thioglutaric anhydride, (as illustrated in FIG. 5 by entries 7 and 8), and the like, many of which are very readily prepared.

The functional group compatibility of the method is highlighted by the use of carbohydrate-based amines either as the nucleophile (FIG. 5, entry 4), or in the form of the sequence terminating sulfonamide (FIG. 5, entries 5 and 6). In particular the absence of protecting groups in the latter example is stressed; compatibility with alcohols is further highlighted by the use of methanol as solvent. The use of the morpholine sulfonamide in entries 7 and 8 of FIG. 5 leads to the formation of tertiary amides, marking a clear difference between conventional azide-based chemistry and the new sulfonamide route.

A further extension of this new multicomponent coupling sequence is highlighted by the reaction of thiomaleic anhydride with a bifunctional nucleophile (i.e., an aminothiol). In this chemistry the softer thiol nucleophile undergoes Michael addition to the electrophilic unsaturated thioanhydride, while the harder amine nucleophile reacts with a carbonyl of the thioanhydride moiety. The overall reaction sequence, which results in the formation of a functionalized benzothiazinone, is completed by trapping of the thioacid with a sulfonamide (Scheme 2), and the only byproduct is 2,4-dinitrobenzenethiol. Similar heterocycle forming systems can employ other bifunctional nucleophiles in this sequence.

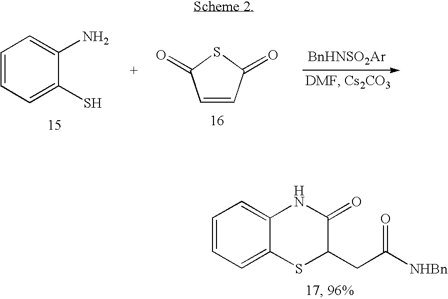

The present method is adaptable for use with more highly functionalized cyclic thioanhydrides, and in particular ones derived from aspartic acid or glutamic acid. In a preferred embodiment, N-Cbz-L-aspartic anhydride was treated with Na₂S according to the procedure of Example 3 and provided compound (20) in 47% yield (Scheme 3).

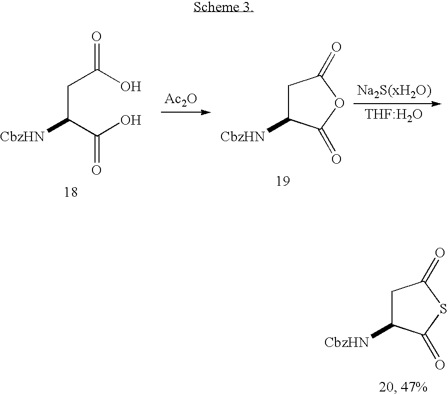

The treatment of compound (20) with aniline in dimethylformamide (DMF) followed by trapping of the intermediate thioacid with the morpholine 2,4-dinitrobenzene sulfonamide (11) afforded the fully functionalized aspartamide (21) in 63% yield as a single regioisomer (Scheme 4), which was confirmed on the basis of Heteronuclear Multiple Bond Correlation (HMBC) experiment. When the reaction was conducted in benzene as solvent, a regioisomeric mixture of products was formed in the ratio of 2.5:1 favoring the formation of compound (21).

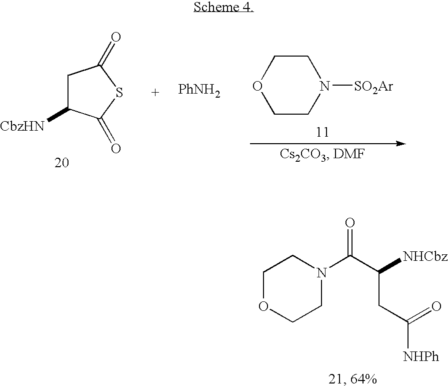

The reaction of compound (20) with 1-glucosamine followed by trapping with the morpholino sulfonamide provided the N-glucosyl asparagine derivative (23) in 44% yield (Scheme 5).

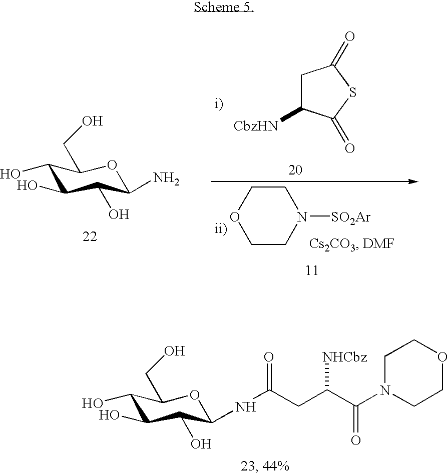

The following non-limiting examples are provided to illustrate certain aspects of the present invention.

### EXAMPLE 1

Synthesis of methyl-N-[(2,4-dinitrophenyl)sulfonyl]-2-amino-2-deoxy-α-D-glucopyranoside (8). About 0.534 g (2.8 mmol, 1.0 equiv.), 2-deoxy-2-amino-methyl-α-D-glucopyranoside was stirred in 10 mL dry dioxane together with 3 mL (about 10 equiv.) pyridine under argon at about −10° C. To this solution 0.810 g (3.0 mmol, 1.1 equiv.) 2,4-dinitrophenylsulfonyl chloride in 5 mL dioxane was added dropwise over 10 min. The reaction was allowed to warm to room temperature, where it was stirred for a further 5 hr., before being cooled to 0° C. and was quenched by addition of 1 mL methanol. The solvent was removed under rotary evaporation and the crude product submitted immediately to column chromatography without further work-up, eluent: EtOAc. Product was isolated as a pale yellow solid (0.714 g, 1.7 mmol, 61% yield), recrystallized from ethanol:hexane, mp: 171.6-172.2. [α]²⁴_{D}: +25.5 (c=1.0, CH₃OH). ¹H NMR (500 MHz, DMSO-d₆) δ 8.81 (d J=2.5 2.5 Hz, 1H), 8.56 (dd J=2.0, 8.5 Hz, 1H), 8.39 (d J=8.5 Hz, 1H), 5.01 (d J=6.0 Hz, 1H), 4.83 (d J=6.0 Hz, 1H), 4.51 (t J=5.5 Hz, 1H), 4.45 (d J=3.5 Hz, 1H), 3.56-3.60 (m, 1H), 3.39-3.44 (m, 2H), 3.24-3.27 (m, 1H), 3.11 (dd J=4.0, 10.5 Hz, 1H), 3.17 (s, 3H), 3.03 (td J=5.5, 9.5 Hz, 1H). ¹³C NMR (125.9 MHz, MeOD): δ 149.7, 147.8, 139.8, 132.3, 126.4, 119.9, 99.2, 72.2, 71.7, 70.8, 61.1, 58.5, 54.3. HRMS (ESI): m/z calculated for C₁₃H₁₇N₃O₁₁ (M+Na)⁺ 446.04763, found 446.0474.

### EXAMPLE 2

Synthesis of 4-[(2,4-dinitrophenyl)sulfonyl]-morpholine (11). About 0.545 mL (6.3 mmol, 1.0 equiv.) freshly distilled morpholine was stirred in 15 mL CH₂Cl₂ together with 0.750 mL (9.4 mmol 1.5 equiv.) at 0° C. under argon. To this mixture 2.00 g (7.5 mmol, 1.2 equiv.) 2,4-dinitrophenylsulfonyl chloride in 10 mL CH₂Cl₂ was added dropwise. The reaction was allowed to warm to room temperature, where it was stirred for a further 5 hr. The reaction was then cooled to 0° C. and quenched by addition of 2 mL of methanol. It was then diluted up with CH₂Cl₂, washed with saturated NaHCO₃ and brine, and dried over Na₂SO₄. After evaporation of solvent the product could be recrystallized from the crude (1.474 g, 4.6 mmol, 74% yield). Yellow solid, recrystallized from EtOAc hexanes, mp 144.1-145.4. ¹H NMR (500 MHz, CDCl₃) δ 8.51 (dd J=2.5 Hz, 8.0 Hz, 1H), 8.47 (dJ=2.0 Hz, 1H), 8.19 (d J=8.5 Hz, 1H), 3.75 (t J=5.0 Hz, 4H), 3.33 (t J=5.0 Hz, 4H). ¹³C NMR (125.9 MHz, CDCl₃): δ 149.9, 14.8, 136.7, 132.7, 126.1, 119.8, 66.4, 46.1. HRMS (ESI): m/z calculated for C₁₀H₁₁N₂O₇S (M+Na)⁺ 360.249001, found 360.2611.

### EXAMPLE 3

N-(benzyloxycarbonyl)-L-aspartic acid thioanhydride (20). A 3.820 g (15 mmol, 1.0 equiv.) of N-(benzyloxycarbonyl)-L-aspartic acid was dissolved in 50 mL CH₂Cl₂ together with 0.050 g (0.15 mmol, 0.01 equiv.) tetrabutylammonium bromide. To this solution was added 1.00 g (7.7 mmol, 0.5 equiv.) Na₂S-xH₂O in 50 mL H₂O. The bi-phasic reaction was stirred vigorously for 3 hr., when the organic layer was separated, the solvent evaporated and the crude product was purified immediately by silica gel chromatography, eluting in 3:1 hexanes:EtOAc (1.911 g, 47% yield based on anhydride). About 1.040 g precipitated as a white solid from diethyl ether:hexane, mp: 87.5-88.4° C. [α]²⁴_{D}: +3.0 (c=1.15, CHCl₃). ¹H NMR (500 MHz, CDCl₃) δ 7.26-7.37 (m, 5H), 6.03 (d J=6.5 Hz, 1H), 5.08 (s, 2H), 4.66-4.71 (m, 1H), 3.25 (dd J=8.5 Hz, 1H), 3.10 (dd J=9.0 Hz, 1H). ₁₃C NMR (125.9 MHz, CDCl₃): 199.2, 195.2, 156.0, 135.7, 128.8, 128.6, 128.4, 128.3, 67.7, 60.4, 45.4. Elemental analysis: calcd. for C₁₂H₁₁NO₄S % C: 54.33, % H: 4.18, % N: 5.28, % S: 12.09; found % C: 54.51, % H: 4.10, % N: 5.24, % S: 12.05.

### EXAMPLE 4

General procedure for multi-component coupling reaction. To a stirred solution of thioanhydride (about 1.0 mmol, 1.2 equiv.) in DMF (about 1 mL, to 1 M in thioanydride) was added 1.0 equiv. amine (about 0.8 mmol) in an equal volume of DMF (about 1 mL) at room temperature. The reaction was allowed to stir for 30min.-1 hr., after which time the color of the reaction had turned a faint yellow. 1.0 equiv. Cs₂CO₃ was added followed immediately by 1.0 equiv. sulfonamide. Upon addition of the sulfonamide the reaction became a dark red, which deepened in color as the reaction continued. The reaction was allowed to stir about 1 hr., after which the DMF was removed under high vacuum and the crude mixture was dissolved in EtOAc and washed with NaHCO₃ and brine and dried over Na₂SO₄. Evaporation of solvent, followed by column chromatography provided the diamide products in yields as described below. In instances where the products were water soluble the extraction was forgone and the crude mixture submitted directly to the column after removal of DMF.

### EXAMPLE 5

N-phenyl-N′-phenylmethyl-butanediamide (5) was prepared according to the general procedure of Example 4, utilizing aniline, succinic thioanhydride, and N-benzyl-(2,4-dinitrophenyl)sulfonamide. Product characterization: pale yellow solid, recrystallized from EtOAc:hexanes, mp: 175.0-178.5. ¹H NMR (500 MHz, CDCl₃/MeOD) δ 7.48-7.50 (m, 2H), 7.23-7.28 (m, 6H), 7.17-7.21 (m, 1H), 7.05 (t J=7.5 Hz, 1H), 4.36 (s, 2H), 2.66-2.69 (m, 2H), 2.58-2.60 (m, 2H). ¹³C NMR (125.9 MHz, CDCl₃/MeOD): δ 173.1, 171.5, 138.2, 128.6, 128.3, 127.3, 127.0, 123.9, 120.0, 43.1, 32.0, 30.9. Elemental analysis: calculated for C₁₇H₁₈N₂O₂ % C: 72.32, % H: 6.43; found % C: 72.47, % H: 6.25.

### EXAMPLE 6

N-Phenylmethyl-N′-(2,3,4,6-tetra-O-acetyl-β-D-glucopyranosyl)-butanediamide (7) was prepared according to the procedure of Example 4 utilizing aniline, succinic thioanhydride, and sulfonamide (8). Product characterization: pale yellow oil. [α]²⁴_{D}: +25.5 (c=0.4, CHCl₃). ¹H NMR (500 MHz, CDCl₃) δ 7.30-7.34 (m, 2H), 7.24-7.29 (m, 3H), 6.73 (d J=9.0 Hz, 1H), 6.05 (t J=5.0 Hz, 1H), 5.29 (t J=9.5 Hz, 1H), 5.24 (t J=9.0 Hz, 1H), 5.06 (t J=10.0 Hz, 1H), 4.93 (t J=9.5 Hz, 1H), 4.41 (d J=6.0 Hz, 2H), 4.28 (dd J=3.5, 12.5 Hz, 1H), 4.06 (dd J=2.0, 12.5 Hz, 1H), 3.78-3.81 (m, 1H), 2.44-2.65 (m, 4H), 2.07 (s, 3H), 2.04 (s, 3H), 2.03 (s, 3H), 2.01 (s, 3H). ¹³C NMR (125.9 MHz, CDCl₃): δ 172.7, 171.4, 171.1, 170.7, 170.0, 169.5, 138.1, 128.8, 128.7, 128.4, 127.7, 127.6, 78.3, 73.5, 72.8, 70.5, 68.1, 61.7, 43.7, 31.4, 30.8, 20.8, 20.7, 20.6. HRMS (ESI): m/z calculated for C₂₅H₃₂N₂O₁₁ (M+H)⁺ 537.20789, found 537.2075.

### EXAMPLE 7

Methyl 2-deoxy-2-[(4-phenylamino-1,4-dioxobutyl)amino]-α-D-glucopyranoside (9) was prepared according to the general procedure of Example 4 utilizing aniline, succinic thioanhydride, and sulfonamide (8). Product characterization: pale yellow oil. [α]²⁴_{D}: +64.8 (c=0.8, CH₃OH). ¹H NMR (500 MHz, CDCl₃/MeOD) δ 7.51-7.54 7.54 (m, 2H), 7.26-7.30 (m, 2H), 7.06 (t J=7.0 Hz, 1H), 4.64 (d J=3.0 Hz, 1H), 3.90-3.95 (m, 1H), 3.82 (dd J=3.0, 12.5 Hz, 1H), 3.64-3.70 (m, 2H), 3.52-3.56 (m, 1H), 3.34-3.37 (m, 4H), 2.65-2.70 (m, 2H), 2.60-2.63 (m, 2H). ¹³C NMR (125.9 MHz, CDCl₃/MeOD): δ 173.8, 171.7, 138.5, 128.4, 123.7, 119.8, 98.5, 72.3, 71.7, 70.8, 61.3, 54.2, 54.0, 31.7, 30.5. HRMS (ESI): m/z calculated for C₁₇H₂₄N₂O₇ (M+Na)+391.14760, found 391.14741.

### EXAMPLE 8

N-(4-morpholinyl)-N′-phenyl-1,1-cyclobutanedicarboxamide (12) was prepared by the general procedure of Example 4 utilizing aniline, 1,1-cyclobutanedicarboxylic thioanhydride (10), and sulfonamide (11). Product characterization: clear oil. ¹H NMR (500 MHz, CDCl₁₃/MeOD) δ 7.42-7.44 (m, 2H), 7.22-7.26 (m, 2H), 7.04 (t J=7.0 Hz, 1H), 3.55 (s, 4H), 3.44-3.46 (m, 2H), 3.27-3.30 (m, 2H), 2.53-2.64 (m, 4H), 1.88-1.94 (m, 1H), 1.79-1.81 (m, 1H). ¹³C NMR (125.9 MHz, CDCl₃/MeOD): δ 171.5, 169.7, 137.9, 128.9, 124.5, 119.9, 66.6, 66.5, 54.8, 46.0, 43.0, 29.5, 15.3. HRMS (ESI): m/z calculated for C₁₆H₂₀N₂O₃ (M+H)⁺ 289.15467, found 289.15444.

### EXAMPLE 9

N-Phenyl-N′-phenylmethyl-pentanediamide (14) was prepared by the general procedure of Example 4 utilizing aniline, thioanhydride (13) and sulfonamide (11). Product characterization: pale yellow solid, recrystallized from chloroform hexanes, mp: ¹H NMR (500 MHz, CDCl₃/MeOD) δ 7.42 (d J=7.5 Hz, 2H), 7.17-7.21 (m, 2H), 6.98 (t J=7.5 Hz, 1H), 3.84 (s, 4H), 3.55-3.58 (m, 2H), 3.48 (−3.50 (m, 1H), 3.40 (t J=5.0 Hz, 1H), 2.29-2.35 (m, 4H), 1.95 (quintet J=7.5 Hz, 1H), S-5 1.88 (quintet J=7.0 Hz, 1H). ¹³C NMR (125.9 MHz, CDCl₃/MeOD): δ 172.3, 171.9, 138.1, 128.7, 124.1, 124.0, 120.0, 119.8, 66.6, 66.5, 46.0, 41.9, 36.0, 31.9, 21.9, 21.1. HRMS (ESI): m/z calculated for C₁₅H₂₀N₂O₃ (M+H)⁺ 277.15467, found 277.15447.

### EXAMPLE 10

[(1S)-3-(phenylamino)-3-oxo-1-[(4-morpholinyl)carbonyl]propyl]-carbamic acid benzyl ester (21) was prepared according to the general procedure, i.e. 0.060 g of thioanhydride (20) was combined with 0.025 mL aniline and 0.060 g of sulfonamide (11) to yield about 0.050 g of (21) as a clear oil. The ¹H NMR spectrum of the compound exhibited a mixture of two sets of signals, which coalesced on heating to 50° C. and diverged on cooling to −20° C., thus demonstrating themselves to be rotamers of a single compound. Spectra from the variable temperature experiment are presented below. [α]²⁴_{D}: +30.6 (c=1.0, CDCl₃). ¹H NMR (500 MHz, CD₃CN, 0° C.) δ 9.25 (s, 1H), 9.17 (s, 0.4 H), 7.46-7.52 (m, 2.8 H), 7.23-7.34 (m, 9.8 H), 7.04-7.09 (m, 1.4H), 6.78 (d, J=7.5 Hz, 1H), 5.19 (d J=11.5 Hz, 0.4 H), 5.10-5.14 (m, 1H), 5.05 (d J =11.5 Hz, 1H), 5.00 (d J=12.5 Hz, 1H), 4.92-4.95 (m, 0.8 H), 3.18-3.69 (m, 11.2 H), 2.89 (dd J=9.0, 15.0 Hz, 1H), 2.70 (dd J=6.5, 15.0 Hz, 1H), 2.55 (dd J=9.0, 14.0 Hz, 0.4 H), 2.38-2.40 (m, 0.4 H). ¹³C NMR (125.9 MHz, CDCl₃, 25° C.): 170.4, 168.1, 155.9, 138.1, 136.1, 129.0, 128.6, 128.3, 128.2, 124.3, 119.7, 67.2, 66.6, 66.5, 47.7, 46.5, 42.9, 40.3. HRMS (ESI): m/z calculated for C₂₂H₂₅N₃O₅ (M+H)⁺ 412.18670, found 412.18629.

### EXAMPLE 11

[(1S)-3-(β-D-glucopyranosylamino)-3-oxo-1-[(4-morpholinyl)carbonyl]propyl]-carbamic acid-benzyl ester (23) was prepared according to the general procedure of Example 4 utilizing (1S)-3-β-D-glucopyranosyl amine, thioanhydride (20), and sulfonamide (11). Product characterization: clear oil. [α]²⁴_{D}: +30.6 (c=1.0, CH₃OH). ¹H NMR (500 MHz, CD₃CN) δ 7.30-7.38 (m, 5H), 5.08 (d J=12.0 Hz, 1H), 5.02 (d J=12.0 Hz, 1H), 4.89-4.95 (m, 1H), 4.82 (dd J=8.0, 9.0 Hz, 1H), 3.71 (dd J=2.0, 12.0 Hz, 1H), 3.40-3.61 (m, 9H), 3.37 (dt J=2.0,9.0 Hz, 1H), 3.30-3.34 (m, 1H), 3.16-3.26 (m, 2H), 2.73-2.78 (m, 1H), 2.48-2.59 (m, 1H). ¹³C NMR (125.9 MHz, CD₃CN): 171.0, 169.7, 155.9, 136.9, 128.5, 128.0, 127.8, 79.4, 77.9, 77.0, 72.4, 70.0, 66.5, 66.2, 61.4, 47.4, 46.1, 42.5, 38.2. HRMS (ESI): m/z calculated for C₂₂H₃₁N₃O₁₀ (M+H)⁺ 498.20822, found 498.20803.

### EXAMPLE 12

3,4-dihydro-3-oxo-N-(phenylmethyl)-2H-1,4-Benzothiazine-2-acetamide (17). About 0.048 g (0.4 mmol, 1.0 equiv) 2-thioaniline was added to a stirred solution of 0.053 g (0.46 mmol, 1.2 equiv.) maleic thioanhydride2 in DMF at 0° C. The reaction immediately turned a dark purple, which deepened as the it was warmed to room temperature. The reaction was allowed to stir for 30 min., then cooled to 0° C. and 0.126 g Cs₂CO₃ was added, followed immediately by 0.130 g (0.4 mmol, 1.0 equiv.) N-benzyl-(2,4-dinitrophenyl)-sulfonamide. The reaction was allowed to warm to room temperature and stirred for a further 30 min. The solvent was then removed under vacuum and the crude dissolved in EtOAc, washed with saturated aqueous NaHCO₃ and brine and dried over Na₂SO₄. Evaporation of solvent left an orange solid, which could then be recrystallized from THF to give the analytically pure product (17) (0.116 g, 96% yield). Pale yellow solid, mp: 238.5-240° C.; lit.₃: 239-240° C. ¹H NMR (500 MHz, DMSO-d₆) δ 8.49 (t J=5.5 Hz, 1H), 7.25-7.35 (m, 5H), 7.22 (t J=7.0 Hz, 1H), 7.19 (t J=7.5 Hz, 1H), 6.97-6.99 (m, 2H), 4.23-4.32 (m, 2H), 3.85 (dd J=5.0, 8.5 Hz, 1H), 2.78 (dd J=5.0, 15.0 Hz, 1H), 2.39 (dd J=9.0, 15.0 Hz, 1H). ¹³C NMR (125.9 MHz, DMSO-d₆): δ 168.9, 166.6, 139.7, 137.3, 129.4, 129.3, 128.7, 128.2, 127.6, 127.2, 123.6, 118.5, 117.5, 42.6, 38.4, 35.5. Elemental analysis: calculated for C₁₇H₁₆N₂O₂S % C: 65.36, % H: 5.16, % N: 8.97, % S: 10.26; found % C: 65.47, % H: 5.27, % N: 8.91, % S: 10.20.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for coupling an amino or hydroxyl compound with the amino portion of a sulfonamide, the method comprising contacting an amino or hydroxyl compound of Formula (I) with a cyclic thioanhydride of Formula (II) for a period of time sufficient to form an intermediate of Formula (III), and contacting the intermediate of Formula (III) with an aryl sulfonamide compound of Formula (IV) in the presence of a base to form a coupled amide of Formula (V), as set forth in the following reaction scheme: 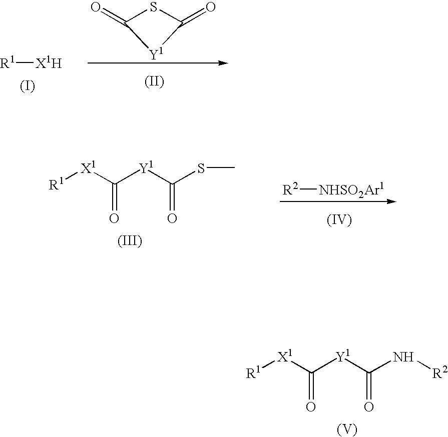
wherein X¹ is —NH— or —O—; Y¹ is a hydrocarbon group forming a 4- to 10-membered ring with the thioanhydride portion of Formula II; Ar¹ is an electron deficient aryl group; and R¹ and R² are each independently a hydrocarbon group, a carbohydrate group, an amino acid group, or a peptide group.

2. The method of claim 1 wherein the cyclic thioanhydride of Formula (II) is 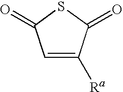
wherein R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; and R^{b} is H, alkyl, arylalkyl, aryl, alkyloxy, arylalkyloxy, aryloxy, alkylamino, arylalkylamino, arylamino, or NH₂.

3. The method of claim 1 wherein the cyclic thioanhydride of Formula (II) is 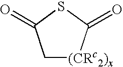
wherein each R^{c} is independently H, alkyl, arylalkyl, or aryl; and x is 1, 2, 3, 4, 5, or 6.

4. The method of claim 1 wherein the cyclic thioanhydride of Formula (II) is 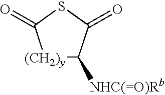
wherein R^{b} is H, alkyl, arylalkyl, aryl, alkyloxy, arylalkyloxy, aryloxy, alkylamino, arylalkylamino, arylamino, or NH₂; and y is 1 or 2.

5. The method of claim 1 wherein the cyclic thioanhydride of Formula (11) is 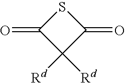
wherein each R^{d} is independently H, alkyl, arylalkyl, aryl, or both R^{d} groups together form —CR^{c}₂—(CR^{c}₂)_{z}—CR^{c}₂—; and z=1, 2, 3 or 4.

5. The method of claim 1 wherein R¹ comprises an amino acid group.

6. The method of claim 1 wherein R¹ comprises a peptide group.

7. The method of claim 1 wherein R¹ comprises a carbohydrate group.

8. The method of claim 7 wherein the carbohydrate group comprises a sugar.

9. The method of claim 7 wherein the carbohydrate group comprises a polysaccharide.

10. The method of claim 1 wherein R² comprises an amino acid group.

11. The method of claim 1 wherein R² comprises a peptide group.

12. The method of claim 1 wherein R² comprises a carbohydrate group.

13. The method of claim 12 wherein the carbohydrate group comprises a sugar.

14. The method of claim 12 wherein the carbohydrate group comprises a polysaccharide.

15. The method of claim 1 wherein X¹ is —NH—.

16. The method of claim 1 wherein Ar¹ comprises an electron deficient substituted phenyl group.

17. The method of claim 1 wherein Ar¹ is 2,4-dinitrophenyl.

18. A method for forming a sulfur-nitrogen heterocycle comprising contacting an aminothio compound of Formula (29) with a cyclic thioanhydride of Formula (24) for a period of time sufficient to form a thioacid intermediate, and contacting the thioacid intermediate with an aryl sulfonamide compound of Formula (IV) in the presence of a base to form a coupled heterocyclic amide of Formula (31), as set forth in the following reaction scheme: 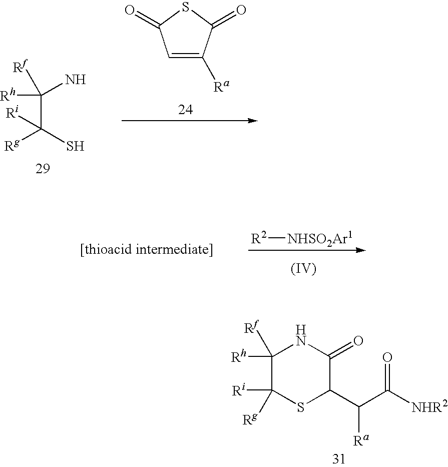
wherein R^{a} is H, alkyl, arylalkyl, aryl, alkyloxy, arylalkyloxy, aryloxy, alkylamino, arylalkylamino, arylamino, NHC(═O)R^{b}, CN, or C(═O)R^{b}; R^{b} is H, alkyl, arylalkyl, aryl, alkyloxy, arylalkyloxy, aryloxy, alkylamino, arylalkylamino, arylamino, or NH₂; Ar¹ is an electron deficient aryl group; R² is a hydrocarbon group, a carbohydrate group, an amino acid group, or a peptide group; and wherein each R^{f}, R^{g}, R^{h}, and Rⁱ independently is H, alkyl, arylalkyl, aryl, or alternatively, R^{f} and R^{g} are H, while R^{h} and Rⁱ together form an aliphatic hydrocarbon ring; or R^{f} and R^{g} are absent, while R^{h} and Rⁱ together form an aromatic hydrocarbon ring.

19. The method of claim 18 wherein Ar¹ is 2,4-dinitrophenyl; and R^{f} and R^{g} are absent, while R^{h} and Rⁱ together form an aromatic hydrocarbon ring.

20. A method for forming an asparagine or glutamine derivative comprising contacting an amino or hydroxyl compound of Formula (I) with a cyclic thioanhydride of Formula (26) for a period of time sufficient to form an intermediate of Formula (32), and contacting the intermediate of Formula (32) with an aryl sulfonamide compound of Formula (IV) in the presence of a base to form a coupled asparagine or glutamine derivative of Formula (33), as set forth in the following reaction scheme: 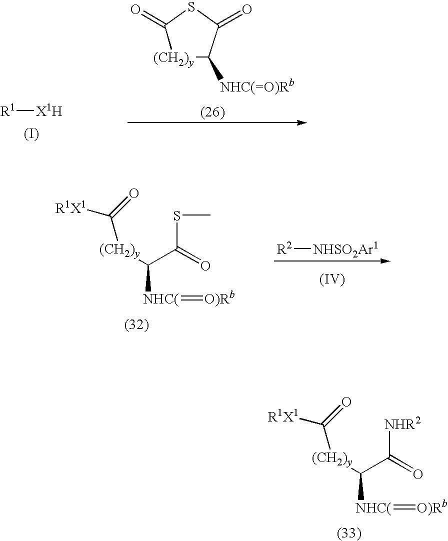
wherein X¹ is —NH— or —O—; Ar¹ is an electron deficient aryl group; R¹ and R² are each independently a hydrocarbon group, a carbohydrate group, an amino acid group, or a peptide group; R^{b} is H, alkyl, arylalkyl, aryl, alkyloxy, arylalkyloxy, aryloxy, alkylamino, arylalkylamino, arylamino, or NH₂; and y is 1 or 2.
